(19) United States
(12) Patent Application Publication
(10) Pub. No.: US 2022/0058749 A1
(43) Pub. Date: Feb. 24, 2022

(54) MEDICAL FRAUD, WASTE, AND ABUSE ANALYTICS SYSTEMS AND METHODS

(71) Applicant: Alivia Capital LLC, Woburn, MA (US)

(72) Inventors: Kleber S. Gallardo, Lexington, MA (US); Matthew K. Perryman, Somerville, MA (US)

(21) Appl. No.: 17/407,810

(22) Filed: Aug. 20, 2021

Related U.S. Application Data
(60) Provisional application No. 63/068,144, filed on Aug. 20, 2020.

(51) Int. Cl. G06Q 40/08; G06N 7/00; G06K 9/62

(52) U.S. Cl. CPC G06Q 40/08; G06K 9/6218; G06N 7/00

(57) ABSTRACT

A method of processing a medical claim includes receiving a medical claim associated with a patient; creating a digital twin of the patient; mathematically analyzing whether the medical claim comports with the digital twin; and outputting an indication of potential claim fraud if the medical claim does not comport with the digital twin.

### CROSS-REFERENCE TO RELATED APPLICATION(S)

This patent application claims the benefit of U.S. Provisional Patent Application No. 63/068,144 entitled MEDICAL FRAUD, WASTE, AND ABUSE ANALYTICS SYSTEMS AND METHODS filed Aug. 20, 2020, which is hereby incorporated herein by reference in its entirety.

The subject matter of this patent application also may be related to the subject matter of U.S. patent application Ser. No. 15/462,312 entitled ANALYTICS ENGINE FOR DETECTING MEDICAL FRAUD, WASTE, AND ABUSE filed Mar. 17, 2017 published Sep. 21, 2017 as US 2017/0270435, which claims the benefit of U.S. Provisional Patent Application No. 62/310,176 filed Mar. 18, 2016, each of which is hereby incorporated herein by reference in its entirety.

### STATEMENT REGARDING PRIOR DISCLOSURES BY THE INVENTOR OR A JOINT INVENTOR UNDER 37 C.F.R. 1.77(b)(6)

Aspects of Dashboard to Dashboard Drill Down (described below) were demonstrated at a conference Aug. 25, 2019-Aug. 28, 2019.

Aspects of digital twin analysis (described below) were provided to customers beginning around December 2020.

Pursuant to the guidance of 78 Fed. Reg. 11076 (Feb. 14, 2013), Applicant is identifying this disclosure in the specification in lieu of filing a declaration under 37 C.F.R. 1.130(a). Applicant believes that such disclosure is subject to the exceptions of 35 U.S.C. 102(b)(1)(A) or 35 U.S.C. 102(b)(2)(a) as having been made or having originated from one or more members of the inventive entity of the application under examination.

### FIELD OF THE INVENTION

The invention generally relates to data analytics and, more particularly, the invention relates to visualizations of data analytics.

### BACKGROUND OF THE INVENTION

U.S. healthcare expenditure in 2014 was roughly 3.8 trillion. The Centers for Medicare and Medicaid Services (CMS), the federal agency that administers Medicare, estimates roughly $60 billion, or 10 percent, of Medicare's total budget was lost to fraud, waste, and abuse. In fiscal year 2013, the government only recovered about $4.3 billion dollars.

### SUMMARY OF VARIOUS EMBODIMENTS

In accordance with one embodiment, a method of processing a medical claim comprises receiving a medical claim associated with a patient; creating a digital twin of the patient; mathematically analyzing whether the medical claim comports with the digital twin; and outputting an indication of potential claim fraud if the medical claim does not comport with the digital twin.

In accordance with another embodiment, a system for processing a medical claim comprises at least one processor coupled to at least one memory storing computer program instructions which, when executed by the at least one processor, cause the system to perform computer processes comprising receiving a medical claim associated with a patient; creating a digital twin of the patient; mathematically analyzing whether the medical claim comports with the digital twin; and outputting an indication of potential claim fraud if the medical claim does not comport with the digital twin.

In accordance with another embodiment, a computer program product comprises a tangible, non-transitory computer readable medium having embodied therein computer program instructions for processing a medical claim which, when executed by at least one computer processor, cause the computer processor to perform computer processes comprising receiving a medical claim associated with a patient; creating a digital twin of the patient; mathematically analyzing whether the medical claim comports with the digital twin; and outputting an indication of potential claim fraud if the medical claim does not comport with the digital twin.

In various alternative embodiments, mathematically analyzing whether the medical claim comports with the digital twin may involve determining a degree to which the medical service comports with the mathematical model and outputting a score indicating the degree. The digital twin of the patient may be created at least in part using a baseline representation of medical codes to mirror a health state of the patient. The medical claim may include a Resource Utilization Group (RUG) categorization for the patient, in which case mathematically analyzing whether the medical claim comports with the digital twin may involve mathematically analyzing whether the Resource Utilization Group (RUG) categorization comports with the mathematical model, which in turn may involve determining a projected RUG categorization for the patient based on the digital twin and determining whether the projected RUG comports with the RUG in the medical claim. Mathematically analyzing whether the medical claim comports with the digital twin may involve determining whether a medical service associated with the medical claim was medically indicated based on the digital twin and/or whether a medical service associated with the medical claim is consistent with medical norms with respect to a health state of the patient.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows examples of single-step visualization of PCA analysis of large-scale data relevant to medical FWA.

FIG. 2 shows examples of the medical FWA-related data visualization of FIG. 1 before stepwise dimensionality reduction (left-hand visualization) and following stepwise dimensionality reduction (right-hand visualization), in accordance with one exemplary embodiment.

FIG. 3 shows a pull-down menu for dashboard drill down, in accordance with one exemplary embodiment.

FIG. 4 shows a drill down destination dashboard, in accordance with one exemplary embodiment.

FIG. 5 shows a Consecutive Drill Down Source Dashboard, in accordance with one exemplary embodiment.

FIG. 6 shows a Consecutive Drill Down Destination Dashboard, in accordance with one exemplary embodiment.

FIG. 7 shows an example of running an ad-hoc process, in accordance with one exemplary embodiment.

FIG. 8 shows an example of ad-hoc execution windowing and filtration, in accordance with one exemplary embodiment.

FIG. 9 shows an example of Galactic Filter, in accordance with one exemplary embodiment.

FIG. 10 shows an example of adding a dashboard to a task, in accordance with one exemplary embodiment.

FIG. 11 shows an example of an opened dashboard, in accordance with one exemplary embodiment.

FIG. 12 shows an example of adding a dashboard to a task via Dashboard, in accordance with one exemplary embodiment.

FIG. 13 is a schematic diagram showing a FWA analytics system having one or more processors that run computer program instructions that cause the system to receive a medical claim associated with a patient, create a digital twin of the patient, mathematically analyze whether the medical claim comports with the digital twin, and output an indication of potential claim fraud if the medical claim does not comport with the digital twin, in accordance with various embodiments.

It should be noted that the foregoing figures and the elements depicted therein are not necessarily drawn to consistent scale or to any scale. Unless the context otherwise suggests, like elements are indicated by like numerals.

### DESCRIPTION OF ILLUSTRATIVE EMBODIMENTS

### Introduction

Exemplary embodiments relate to a Health Care Fraud Waste and Abuse (FWA) predictive analytics system. As such, exemplary embodiments provide technological solutions to problems that arises squarely in the realm of technology. Applicant believes that such solutions are not well-understood, routine, or conventional to a skilled artisan in the field of the present invention.

In illustrative embodiments, the FWA predictive analytics system is a browser-based software package that provides quick visualization of data analytics related to the healthcare industry, primarily for detecting potential fraud, waste, abuse, or possibly other types of anomalies (referred to for convenience generically herein as “fraud”).

Users are able to connect to multiple data sources, manipulate the data and apply predictive templates and analyze results. Details of illustrative embodiments are discussed below with reference to a product called FWA FINDER™ (formerly Absolute Insight) from Alivia Analytics of Woburn, MA, some features of which are described in U.S. patent application Ser. No. 15/462,312 entitled ANALYTICS ENGINE FOR DETECTING MEDICAL FRAUD, WASTE, AND ABUSE filed Mar. 17, 2017 published Sep. 21, 2017 as US 2017/0270435 (hereinafter referred to as “the Analytics Engine Patent Application,” which was incorporated by reference above), and in which various embodiments discussed herein are or can be implemented.

FWA FINDER™ is a big data analysis software program (e.g., web-browser based) that allows users to create and organize meaningful results from large amounts of data. The software is powered by, for example, algorithms and prepared models to provide users “one click” analysis out of the box.

In some embodiments, FWA FINDER™ allows users to control and process data with a variety of functions and algorithms and creates analysis and plot visualizations. FWA FINDER™ may have prepared models and templates ready to use and offers a complete variety of basic to professional data messaging, cleansing and transformation facilities. Its Risk score and Ranking engine is designed so that it takes about a couple of minutes to create professional risk scores with a few drags and drops.

In some embodiments, the data analysis software provides benefits including:
Unobtrusive. For example, the software may be browser-based with zero desktop footprint.
Deep Intelligence that allows the user to understand why things are happening
Predictive Intelligence: predict what will happen next
Adaptive Learning: system learns and adjusts based on actual results
Complete Analytics Workflow: intuitive analytics processes
Powerful Insights: immediate productivity gains with drag and drop
Data Science in a Box: quickly understand the significance of the data
Perceptive Visualizations: articulate analysis with meaningful visualizations
Seamless Data Blending: quickly connect disparate data sources
Simplified Analytics: leverage prebuilt analytic models
Robust Security: be confident your data and analysis are secure

To that end, in some embodiments, FWA FINDER™ provides cloud-enabled pre-built data mining models, predictive analytics, and distributed in-memory computing.

### Data Normalization, Ranking, and Enrichment

As discussed in the Analytics Engine Patent Application, exemplary embodiments provide a ranking capability for data preparation and manipulation. Features range from basic sorting, filtering, and adding/removing attributes/columns, to exclusive features like creating new combined columns, re-weighting attributes, assigning ranks to each record to detect anomalies/patterns, and creating more informative views of data from the data source. In certain embodiments, each type of data (e.g., each column of data to be used in an analysis or model) is normalized to a value between 0 and 1, e.g., by assigning a value of 0 to the minimum value found among the type of data, assigning a value of 1 to the maximum value found among the type of data, and then normalizing the remaining data relative to these minimum and maximum values. In this way, each relevant column has values from 0 to 1. Values from multiple columns can then be “stacked” (e.g., added) to come up with a pseudo-risk score.

In further exemplary embodiments, the system can work on multiple data sources from both clients and the outside world, both free and paid, e.g., CSV or other files, e.g., medical records, divorce data, financial data, personal data, etc. The system can harmonize data by connecting different pieces of information together, e.g., using a key such as a provider identifier that can be used to pull data from the various data sources. In order to improve execution speed, the system typically limits the amount of data pulled, e.g., there may be 3000 data fields but perhaps only 100 are needed 80% of the time so the system might only pull those 100 unless others are needed.

This information can be used in analytics, e.g., to evaluate financial stresses or other risks that can lead to fraud. For example, a doctor or patient who is under financial stress such as when going through a divorce or due to large debts (e.g., credit card debt, gambling losses, etc.) might be considered more likely to take fraudulent actions, and such doctors and patients can be flagged for additional scrutiny or monitoring.

The system also can evaluate sources of income for doctors, e.g., is a particular doctor getting paid by a particular drug company or receiving kickbacks or other perks, or is the doctor prescribing a particular medication to the exclusion of other options because it is financially beneficial.

The system also can enrich the data by creating new data points and categories that can be used in the analytics. For example, the system can compute and store distance information, e.g., distance of a patient to a doctor or pharmacy based on latitude/longitude of addresses. Such distance information can then be used in analytics, e.g., the system might flag as suspicious a patient traveling a large distance to a particular doctor or pharmacy, particularly when certain types of activities are involved, e.g., opioid prescriptions. For another example, the system can create summary categories, e.g., does a claim involve an opioid (e.g., opioid yes or no), does a claim involve an ADHD medication (e.g., ADHD yes or no), does a claim involve a brand vs. generic medication, etc. Such summary categories can then be used in analytics and can simplify certain analyses, e.g., multiple claims involving opioids can be viewed as being similar even if they involve different opioids in different doses of both generics and name-brands where otherwise the claims might appear to be dissimilar.

Generally speaking, the system analyzes claim integrity, e.g., was a claimed procedure actually performed, and was a claimed procedure medically necessary, etc.

### Use of “Digital Twins” in Fraud/Waste/Abuse Analysis

It is well-known in the medical care industry to use so-called “digital twins” to help diagnose and predict diseases in patients and patient populations. Generally speaking, a “digital twin” is a mathematical model that can be created for a patient or patient population based on any of various sources of data including, without limitation, medical records, medical claims data, data from IoT devices (e.g., medical devices, wearable physiological and health measuring devices, etc.), disease progression data, etc. The mathematical model can be used to evaluate the current and predicted progression of a patient or patient population condition.

In various exemplary embodiments, “digital twins” are used to model various actors as well as interactions between actors for use in the analysis of fraud, waste, and abuse. These digital twins are then used to assess the likelihood of them belonging to a given clinical group, e.g., Resource Utilization Group (RUG), which dictates facility reimbursement. Facilities with a high propensity to assign groups to patients that are incongruent with their digital twin can be considered “at risk” for “upcoding” their patients (e.g., not accurately assessing and/or treating patients, such as by providing unnecessary rehabilitation, inflated ADL scores given the patient's health, restorative nursing services provided without medical need, etc.), making them seem sicker than they actually are to increase the facility's reimbursement. Specifically, RUG IV categories should relate to the clinical state of the patient. For example, rehabilitation should be more common for post-op patients, while Special Care High, Special Care Low, and Clinically Complex criteria all relate directly to different clinical states and treatments (e.g. diabetes, burns, chemotherapy). Given this, the patient's claiming history should help to substantiate and back up the RUG. One approach, then, is to detect providers who frequently assign RUGs that conflict with the patient's state of health. These providers are likely assessing patients (and potentially providing care) to maximize their reimbursement, regardless of if the care is necessary or appropriate.

The following is the basic process, in accordance with one exemplary embodiment:

1. Create baseline representation of medical codes that can be used to create a mathematical representation of a patient's health state, e.g., use International Statistical Classification of Diseases and Related Health Problems (ICD) codes, National Drug Codes (NDCs), and Current Procedural Terminology (CPT) codes to define “digital twins” of patients that mirror their health state in a mathematical format.

2. Create mathematical representation of patient's health state at the time of the assessment that a model can use to “assess” the patient.

3. Train the model to assess patients given their underlying clinical state.

4. Reassess all patients in (as if the model was the provider) to get a prediction for the RUG/overall index for each assessment.

5. Measure the impact to the RUG when compared to the actual RUG.

6. Rank providers by the overall difference between their RUGs and the most clinically appropriate RUG.

In addition to creating digital twins for patients and/or patient groups, certain exemplary embodiments create digital twins for other types of actors and also for interactions between actors. Digital twins can be created for virtually any and every type of actor involved in medical claims processing, including, without limitation:
Individuals such as patients, providers (e.g., doctors, nurses, etc.), and people who work for healthcare organizations (e.g., investigators, administrative staff, auditors, etc.), e.g., modeling anticipated procedures for patients and drugs they should be taking;
Organizations (e.g., hospitals, clinics, doctor groups, insurance companies, etc.);
Payor Systems (e.g., Adjudication, Eligibility, Provider Enrollment, Enterprise Resource Planning/ERP and inventory control, General Ledger, Customer Relationship Management/CRM, etc.);
Provider Systems (e.g., Claim submission, ERP and inventory control, General Ledger, CRM, Instruments, etc.);
Ecosystems (e.g., Observed interactions such as can be inferred directly from data and Unobserved interactions such as can be inferred from the model of the ecosystem); and

Machines (e.g., there could be a machine such as a computer system acting between other actors, and the system can model the machine).

Also, certain exemplary embodiments create digital twins using a wider range of data sources including data sources that have not traditionally been used in the context of FWA analysis, including, without limitation:
Government data sources (both open and private);
Electronic Health Records (HER);
Claims
Healthcare payor and provider data warehouses;
IoT data (e.g., medical devices, Fitbits, wearables)
Financial records (e.g., certain institutions must provide financial information such as if they service Medicare/Medicaid, individual financial records can expose financial pressures, etc.)
Demographic data (e.g., address/location information, age, etc.);
Legal proceedings and prison records (e.g., a person who has been prosecuted for fraud in the past might be more likely to commit fraud in the future);
Divorce records (e.g., divorce can put financial pressure on an individual and lead to fraudulent behavior, and divorce records can reveal things like net worth, bank account balances, assets, loans, credit card debt, gambling debts, etc.);
Social media;
Licensing (e.g., a person who has been barred in one state might move to another states, a person who has been found fraudulent in one division might move to another division such as from welfare division to Medicaid division or from dental division to medical division, a person who has been found fraudulent in one company/carrier might move to another company/carrier, etc.);
Exclusions;
Genomics;
Phone call records (e.g., to identify the use of so-called “burner” phones often used in fraud schemes based on carrier and phone type, to identify latent connections between people, etc.);
Text messages;
Corporate data (e.g., incorporations, mergers or proposed mergers, can model behavior of fraud rings such as multiple companies that show similar patterns of behavior and have a common executive or related executives such as two college buddies who might be sharing fraud schemes, can model fraudulent claim schemes such as using multiple companies to process more claims than otherwise would be permitted, etc.);
Healthcare regulations (e.g., can model how proposed changes will affect behaviors via the digital twins);
Contract provisions (e.g., can model how proposed changes will affect behaviors via the digital twins, can model if contract provisions are being followed, etc.);
Education (e.g., to confirm qualifications of a doctor, to evaluate socioeconomic status as an indicator for healthcare, etc.);
Resumes;
Emails (e.g., can analyze to detect patterns that might suggest fraudulent behavior);
Web search history (e.g., fraudsters often research fraud schemes, can detect if a person who researched a particular fraud scheme is using that fraud scheme);
Business financials;
Home health testing (e.g., information such as from genetic tests and other home health testing will increasingly be available for incorporation into digital twins);
Reviews and complaints for doctors and other actors (e.g., reviews can provide a good indication of how a doctor acts, such as if patients report that the doctor does not spend enough time or orders too many tests);
Ancestry and relatives (can provide insight into possible current and future medical conditions);
Death history such as from SSA database (e.g., can use to detect certain types of fraud);
Birth certificates;
Eligibility information (e.g., can be used to cross-reference various forms of self-reported behavior such as income);
Organizational and HR data (e.g., can model all employees in a given company who might have the opportunity to commit fraud);
IRS records;
Vehicle and home ownership;
Centers for Medicare and Medicaid Services (CMS) quality metrics (e.g., can be computed for each provider)
Travel information such as from homeland security (e.g., evaluate if a provider was billing even when out of town);
Weather information (e.g., can predict certain types of medical claims based on weather conditions, can evaluate if a provider was billing even when closed due to weather, etc.);
Food and agriculture (e.g., where a person shops for food can be an indicator of future health, what food a person buys can be an indicator of future health, etc.); and
Medical resellers (e.g., checking online sites such as Craigslist or eBay for someone who is selling medical equipment that may have been obtained fraudulently, and correlating based on seller name or phone number).

It should be noted that the model for a given actor may be created from a history of information associated with that actor as well as from a history of information associated with similar individuals and groups. For example, the model for a patient having a particular medical condition can be created using information from others who have had the same or similar medical condition.

It also should be noted that information used in creating digital twins can include virtual information such as from “virtual sensors” that infer information about things that are not actually measured, e.g., through inferences drawn from other data sources. For example, without limitation, a virtual sensor can infer information from social media such as a person's race, religion, sexual orientation, political leanings, risk-taking (e.g., extreme sports, online dating and so-called “hook-up” sites, etc.), schedule, habits, etc. Such information may be used, for example, to evaluate how people use the health care system and how they might react to certain changes in healthcare coverage or laws.

Without limitation, some applications of a healthcare digital twin ecosystem include identifying new and emerging fraud schemes, tracking spread of fraud schemes across the ecosystem (e.g., due to proximity of actors and relationships between actors), inferring unobserved interactions (e.g., inferring kickbacks, for which there will not be actual records showing kickbacks), determining optimal ways to spend healthcare dollars such as to maximize population health, predicting disease outbreaks and how they might spread, disease imputation (e.g., predicting a population that is actually sick from a particular disease, such as inferring a population who have hepatitis C but haven't yet been diagnosed), risk modeling for pricing of insurance (e.g., can analyze overall risk in a particular zip code or based on a person's job), generation of claims, validation of claims such as by evaluating whether a particular claim is consistent with relevant digital twins (e.g., does the claim comport with a patient's modeled condition, with data from various IoT devices, and with the provider's modeled schedule), evaluating and projecting quality of care and patient experience (e.g., evaluating likely outcomes, such as diagnoses tend to go away for Doctor X but tend to remain for Doctor Y, which could indicate that Doctor Y either provides poor quality care or is engaging in fraud by extending a care regimen), generating novel fraud schemes that show weakness of current controls and regulations, predicting effect of implementing new controls and regulations such as on population health and financials (e.g., new drugs, procedures, coverages, etc.), predicting cost impact of covering new services, optimization of operations of payor or provider, predicting effect of mergers and acquisitions for payors and providers such as on population health and quality of service and financials, and differentiation of fraud from waste and abuse such as by identifying intent or lack thereof.

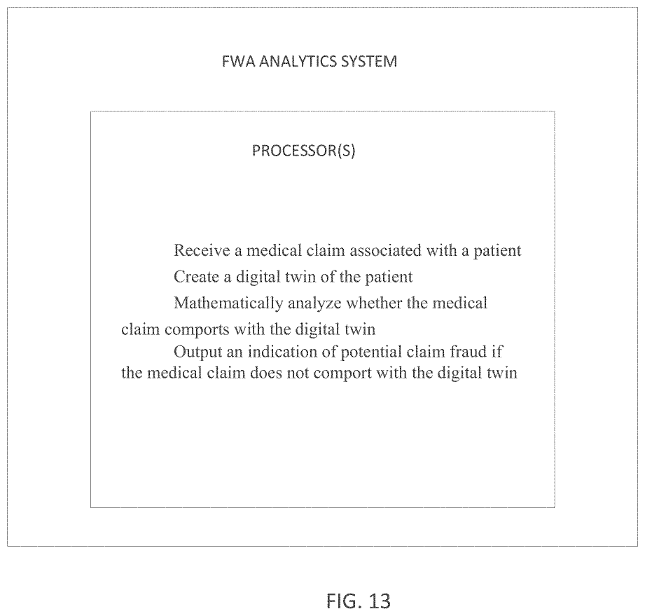

FIG. 13 is a schematic diagram showing a FWA analytics system having one or more processors that run computer program instructions that cause the system to receive a medical claim associated with a patient, create a digital twin of the patient, mathematically analyze whether the medical claim comports with the digital twin, and output an indication of potential claim fraud if the medical claim does not comport with the digital twin, in accordance with various embodiments.

It is envisioned that exemplary FWA analysis systems will typically model many thousands of digital twins to cover the many actors and actor interactions within the ecosystem and that the digital twins will be updates on an ongoing basis, e.g., taking into account new data sources including other digital twins.

### Use of Electronic Medical Records in FWA Analysis

Electronic Medical Records (EMR) are an extremely rich source of information that have already been leveraged to build machine-learning models for predictive diagnosis, hospital readmission, and clinical outcome prediction. EMR contains information in the form of both structured and unstructured data. The former mainly consists of diagnosis codes, procedure codes, drug codes, and test results, while the latter typically consists of doctor's notes and image results (X-Rays, CT Scans). These data points combine to present a rather holistic view of the patient's state of health at a given point in time. Providers interpret this information to make clinical decisions.

After a provider performs a service, they typically submit a claim for payment. These healthcare claims are generally evaluated by adjudication systems that do not evaluate the claim in the context of the medical record. One reason for this is the difficulty of ingesting EMR data, which is often messy and hard to harmonize into a composite picture. Instead, the adjudication system relies on high fidelity coding, which is the process of translating the service performed into the appropriate medical code(s). This gap allows for the possibility of fraud, waste, and abuse (FWA), for example, if the provider codes for something they did not actually perform or that should not have been performed based on the patient's state. For most FWA investigations, one of the first steps of the investigation is to request the medical records to assess if the service was performed as billed and if it was medically necessary. This, however, presumes that the FWA will be flagged for further investigation in the first place (which might not happen if claims are within certain parameters) and also provides an opportunity for the retroactive falsification of medical records (e.g., the provider adding comments that support a claim).

Thus, in certain exemplary embodiments, the system connects directly to the electronic medical records at the time of claim submission, which, among other things, allows the system to validate that the corresponding claim represents a service that actually was performed (e.g., if claim says that an MRI was given, then the system can confirm whether or not an MRI was actually given because there should be an MRI record), validate that the service performed was medically necessary and consistent with the patient's condition and diagnosis, and validate that the medical record is consistent with medical norms. This analysis using EMRs can utilize relevant digital twins such as for the patient and the doctor. Among other things, this use of EMRs will act as a prepayment solution to prevent FWA from being paid as frequently. For example, by using EMRs in this way, a fraudulent claim generally would require a full misrepresentation of the medical record that comports with the submitted claim. This is much more difficult than simply falsifying a claim, which only requires a few codes and a patient ID. Leveraging the EMR in tandem will provide a much higher degree of payment integrity.

The following is the basic process, in accordance with one exemplary embodiment:

1. Ingest medical record and claim

2. Create digital twins for patient, provider, and various coding systems (e.g., ICD diagnoses codes, CPT codes, NDC codes, etc.)

3. Check for mathematical comportment of patient's digital twin state with provider's digital twin state and service(s) claimed

4. Score the claims for their mathematical comportment

5. Rank the claims by their score

6. Set automatic thresholds for prepayment review or rejection

### Cluster Analysis Using Formal Element (FOREL) Analysis

As discussed in the Analytics Engine Patent Application, exemplary embodiments include an Analysis Module that is specially designed to audit, investigate, and find hidden patterns in large amounts of data. It equips the user with the ability to identify patterns in data in just few clicks, and with a list of operators and templates which can help identify fraud, waste or abuse by few drag-and-drops.

Unsupervised Clustering is widely used in data science to infer patterns from relative distances between objects in multi-dimensional space. The space is defined by measurable or estimated properties of the objects (units) selected for the analysis. In the analysis of Medical and Healthcare Fraud, Waste, and Abuse (FWA), clustering is becoming a useful tool to find associations between different fraud schemes, practitioners, patients, and other data objects in the analysis. There are thousands of algorithms and variations available for cluster analysis. However, those different algorithms are based on different assumptions, have different goals and areas of application, and may be useful for a different measure in each situation. Most popular algorithms are hierarchical clustering algorithms that build and trim a tree graph (e.g., a dendrogram) of relations between objects in analysis. Another popular family of algorithms is derived from the k-means method that searches for a given number (k) of high-density areas in the space defined by the traits of the objects in analysis.

In addition to the classic methods for Cluster Analysis, exemplary embodiments can implement an unsupervised clustering algorithm from the FOREL (FORmal ELement) family. The FOREL family of algorithms has been known since the late 1960s and was originally used for statistical data processing in paleontology but was more recently and practically applied to modern High-Performance Computers (HPC). The algorithms of the FOREL family are based on the Natural Taxonomy strategy that does not require the assumption of the existence of cluster structure, specific distribution of object properties, or even the possibility of classifying all objects in a given data set. The clustering is based on a milder assumption of non-uniform distances between objects and therefore “naturally” similar objects being found on relatively shorter distance from each other compared to naturally dissimilar objects. The algorithms of this family are computationally demanding but have low sensitivity to high dimensionality and often provide results that are hard to achieve with other algorithms.

Like other clustering algorithms, FOREL requires definition of the space metric and the distance metric. In addition, FOREL algorithms need a “cluster eminence” or quality metric that can rank possible associations between objects and identify one such association (cluster) as better than the other. Clusters are found and extracted from the data in order of decreasing eminence, best clusters first, until no unclassified objects remain, or the remaining objects do not satisfy the minimal standard for cluster eminence. The metric of cluster eminence defines the specific algorithm within the family. Without limitation, this metric can be based on connectivity within the cluster, density of the cluster, weighted or unweighted distances between members and centroids, etc. Unlike other methods, FOREL can easily distinguish clusters partially or completely overlapping in space, as well as clusters of different density. In multiple tests, FOREL algorithms demonstrated superiority over other approaches to classify medical practitioners by their pattern of participation in fraud schemes. Compared to hierarchical and k-means algorithms, FOREL results can be more meaningful and easier to interpret in certain situations.

A Study of Clustering Algorithm Applications in RBF Neural Networks by P. S. Grabusts available at

http://citeseerx.ist.psu.edu/viewdoc/download?doi=10.1.1.20.3621&rep=rep1&type=pdf, which is hereby incorporated herein by reference in its entirety, describes exemplary FOREL algorithms in which a set of m objects (which can be described by quantitative characteristics, e.g., Euclidian distance in metrical space) can be divided into k taxons (k<m) in different ways. Certain criteria F can be used to distinguish between good and bad groupings and select the best taxonomy variant. FOREL algorithms use the F criterion, which is based on the hypothesis of compactness, e.g., the objects belonging to the same taxon are situated close to each other as compared to the objects belonging to different taxons. As a result, taxons can be derived, e.g., of a spherical shape. The objects included in the same taxon are assigned to a “hyper sphere” with a certain center C and radius R. By changing the radius, the system can derive different number of taxons.

If radius R is fixed, the algorithm can be executed as follows. Center C is placed at any point of the set of objects. Then the points that are inside the sphere are identified. For this purpose, distances d from point C to all M are calculated. Those points for which d<==R are considered as internal to the sphere. The center of gravity for internal points is calculated and the center of sphere is then moved to this center of gravity C. For the new position, internal points and their centers of gravity are found again. The procedure is repeated until the co-ordinates of the gravity center C start varying. This sphere is now called taxon S and its points are excluded from further consideration.

After that, the center of a hyper sphere of the same radius is moved to any of the remaining points and the procedure of taxon revealing is repeated until all the objects are distributed among taxons. Generally speaking, the smaller the taxon radius, the larger the number of taxons. The desired number of taxons for the user can be determined by fitting the radius R properly.

The inventors believe that this is the first application of FOREL algorithms to the analysis of FWA because they are relative obscure clustering algorithms that heretofore have been applied in very specific situations and give different results than more common clustering algorithms. The inventors recognized that FOREL algorithms can be applied to FWA analysis in part because of their ability to separate nested clusters that overlap in space partially or completely and assign seeming outliers to clusters, which in turn can bring focus onto otherwise seemingly unrelated data. Generally speaking, in FWA analysis, the system does not have a priori knowledge of the scale of fraud (or even if any fraud has been committed), the qualities of classes, and the connections between parties and data (e.g., between doctors, patients, etc.). In many cases, events that would be considered a “singleton” or outlier in other clustering algorithms are assigned to a cluster in FOREL, therefore allowing them to be better analyzed in context.

### Stepwise Dimensionality Reduction Following Cluster Analysis For Visualization and Further Analysis

As discussed in the Analytics Engine Patent Application, exemplary embodiments perform dimension reduction, classifier, regression and clustering attempting to mimic human brain modeled by neurons and synapses defined by weights.

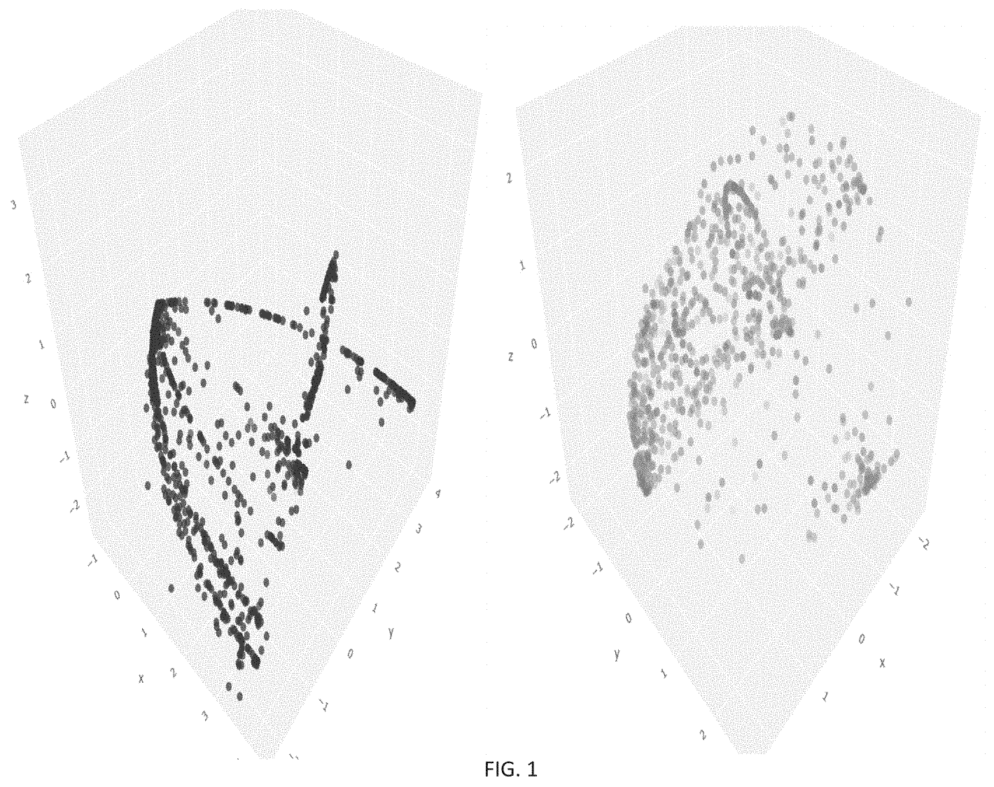

As discussed above, clustering is widely used in data analysis and recently became a useful technique to identify fraud, waste, and abuse (FWA) in Healthcare. Exemplary embodiments can use unsupervised clustering to identify groups of subjects (such as medical practitioners) sharing relevant traits, such as behavioral patterns associated with fraud. The result of such clustering is a list of objects with corresponding cluster number or a list of clusters with members. Visualization and further analysis of cluster properties typically requires further dimensionality reduction down to three (for depiction of cluster juxtaposition in space) or similar small numbers to analyze specific factors contributing to formation of clusters or responsible for difference between clusters. Most typically, Principal Component Analysis (PCA), Factor Analysis (FA), or Singular Value Decomposition (SVD) are the methods applied. Since the number of objects for visualization after PCA or similar dimensionality reduction is still large, plotting the original objects, even with cluster labels and in low-dimensional space, may not reveal their relation patterns, for example, as shown in FIG. 1.

Therefore, in certain exemplary embodiments, the system can perform dimensionality space reduction in a stepwise basis in order to reduce dimensionality to a predetermined level, e.g., to facilitate visualization or for further analysis. The following is an overview of stepwise dimensionality reduction following the cluster analysis (e.g., unsupervised clustering), in accordance with one exemplary embodiment.

In the first step, the system identifies anchor points. Generally speaking, an anchor point is something that characterizes the cluster as an entity, e.g., the centroid of the cluster or the most typical object of the cluster. For example, anchor points can be representative data objects (such as particular medical practitioners) or abstract points in the same feature space adequately representing the class of objects (such as a typical medical practitioner or a centroid of a cluster of medical practitioners). Once the system selects representative anchor points for all objects (clusters, singletons, and such), the system reduces the space to the number of dimensions adequately representing relative distances between those objects. All other objects are identified by the class identity, such as membership in specific cluster. For visualization purposes, all members of the same clusters are assumed to be contained in the space not exceeding the distance from the selected class anchor point to the specific object. Therefore, all objects that belong to the same cluster can be represented by a shape that covers all cluster objects, such as, for example, a sphere with the center at the anchor point (e.g., cluster centroid) and a radius equal to the distance between the anchor point (e.g., cluster centroid) and the most distant object that belongs to that cluster such that the size of the sphere shows how similar the members of the cluster are, e.g., a small sphere indicates that elements are closely related. In this way, the system can provide a 3D display of relationships.

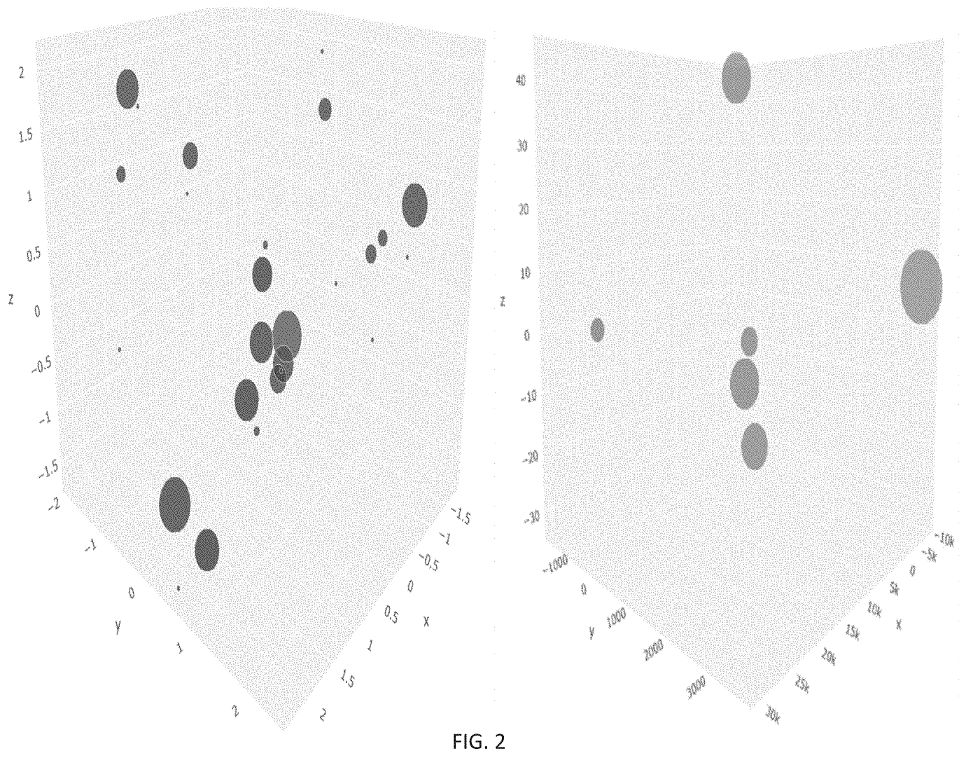

In the second step, the system performs one of the standard techniques for dimensionality reduction (e.g., PCA, FA, or SVD) iteratively (e.g., two or more times if needed) to reduce the dimensionality to a predetermined level (e.g., two, three, or more), depending on specific properties of the data and the requirements for visualization. One practical advantage of stepwise dimensionality reduction is in correct rendition of the geometric properties of objects in feature space without over-cluttering and loss of informative properties, for example, as shown in FIG. 2, which shows examples of the same medical FWA-related data visualization before stepwise dimensionality reduction (left-hand visualization) and following stepwise dimensionality reduction (right-hand visualization), where each cluster is depicted as a sphere with radius proportional to the distance from the cluster centroid (which also are used here as the anchor points) to the most dissimilar member of that cluster. Objects (e.g., clusters, singletons, groups of clusters, etc.) that are related turn out on a short distance from each other; dissimilar objects and groups of objects are on a greater distance. The distance between objects can be deconvoluted into weight factors describing the importance of specific original traits in formation of specific patterns.

### Dashboard to Dashboard Drill Down

As discussed in the Analytics Engine Patent Application, exemplary embodiments include top-of-the-shelf visualization tools that allow for plotting data, including results, to make them more meaningful, presentable and convincing. The visualizations can further be integrated into dashboards to make full investigation/audit reports. Dashboard is used to present analysis work done on data and final results. It also holds Model execution results as well as rule execution results, which can also be used to make a dashboard. Dashboards can be saved as well. In order to add a grid or a chart to a dashboard, the user can select any Model/Rule execution item from “Dashboard & Execution History” of Dashboard. All related results of that particular item will be displayed on right side of dashboard. The user can double-click on any item which is a grid or chart, and it will open a box window in the center of the dashboard. The box window can be resized and dragged anywhere in the center area. This way, all items can be positioned to a suitable location.

Also discussed in the Analytics Engine Patent Application is creating a drill-down tree map plot. Tree maps display hierarchical data by using nested rectangles, that is, smaller rectangles within a larger rectangle. The user can drill down in the data, and the theoretical number of levels is almost unlimited. Tree maps are primarily used with values which can be aggregated. Tree map charts are easy to create, e.g., by dragging and dropping descriptors into columns and dropping values in rows. The user can add multiple descriptors in chain to create a dynamic drillable chart. The user can click on an element such as “Worcester,” in which case the application will drill down to explore all Worcester physicians. Each chart includes a back button, which allows the user to drill back up through a chain of charts.

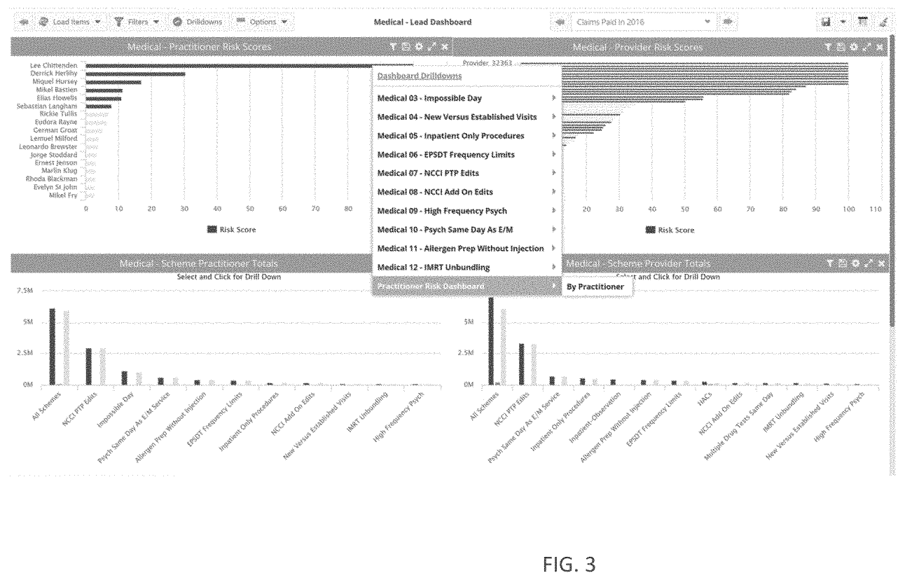

Typically, drill down allows the user to drill down on a chart to expose a table of summary information. This is not the case for the Dashboard to Dashboard drill down as shown, which enables a hierarchy of dashboards. It allows the user to select (e.g., right click) on an entity in a chart and pull up a menu (e.g., a drop-down menu) of other dashboards to drill down to, for example, as shown in FIG. 3. In certain exemplary embodiments, the system evaluates dashboards relating to the given entity to identify dashboards that contain information that is relevant to the particular task and then presents or highlights these dashboards (links) in the user interface, e.g., by only displaying such links or by showing such links along with links to other entity dashboards but then making the other links disappear quickly to indicate to the user that they were checked for the given entity but did not have any results for their selection. This evaluation is done dynamically such that, for example, different sets of dashboards (links) may be presented to the user at different times for a given entity as data is evaluated by the system and dashboards are updated by the system. Thus, the dashboard drill down selections can dynamically change from period to period based on the results generated that drives the dashboard.

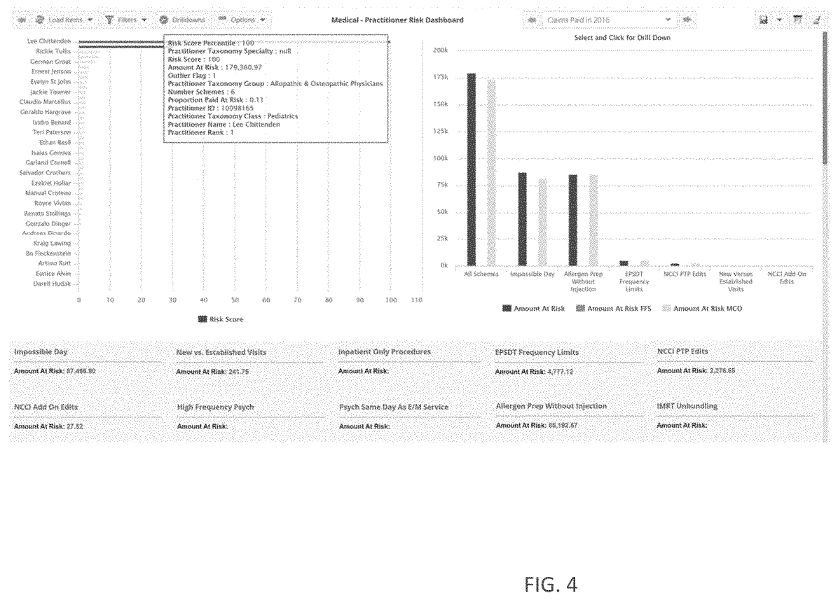

Clicking an option will pull up the selected dashboard filtered for a column or combination of columns associated with the entity chosen in the previous menu, for example, as shown in FIG. 4.

This filtration can be different chart-by-chart. In FIG. 4, the top-left chart is filtered for the Practitioner Taxonomy Group associated with the selected practitioner (e.g., Lee Chittenden), while the top-right chart is filtered for the Practitioner ID associated with Lee Chittenden.

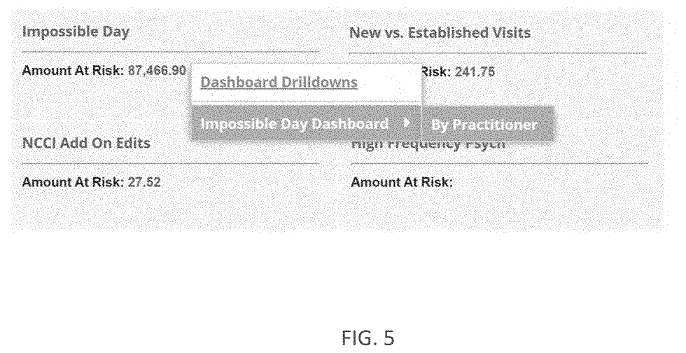

These drill downs can compound on one another, for example, as shown in FIG. 5.

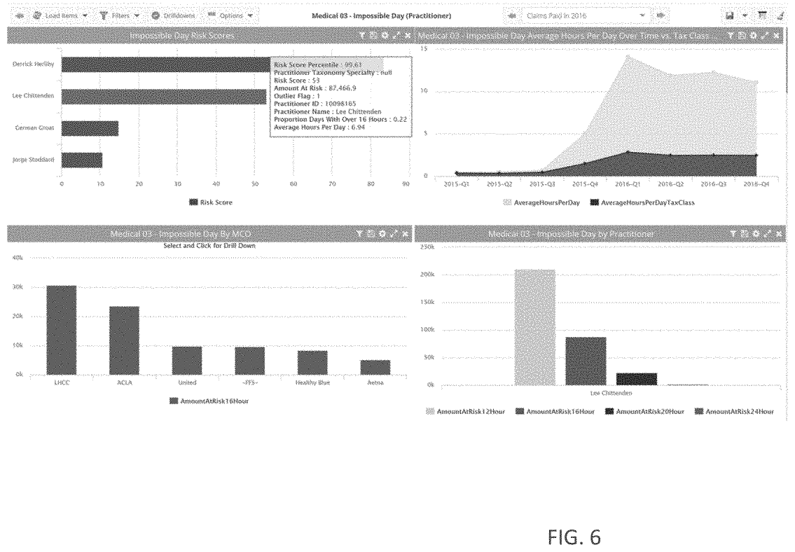

Right clicking the square titled “Impossible Day” (as shown in FIG. 5) allows the user to drill down to a third dashboard to show Dr. Chittenden's results for this specific option selected, for example, as shown in FIG. 6.

This makes the process seamless and user-friendly, as the user is exposed to additional relevant information only when necessary.

Clicking the back button in the top-left of a dashboard will take the user back to the dashboard they were previously viewing in the same state. The hierarchy of dashboards and their associated data can be stored for future reference, such as for providing a chain of evidence in an FWA investigation or trial.

Process Scheduler with Galactic Filter

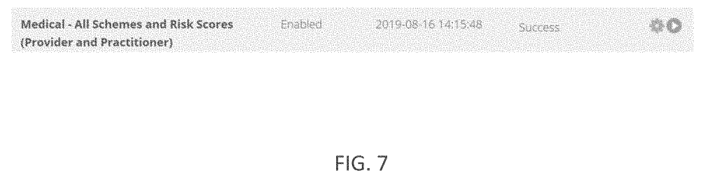

The Process Scheduler allows users to run processes on a schedule or on an ad-hoc basis. These processes can be composed of any FWA FINDER™ Rules, Models, or other Processes. Dependencies are tracked between processes. An example of running the Process “All Schemes and Risk Scores (Provider and Practitioner)” ad-hoc is shown below in FIG. 7.

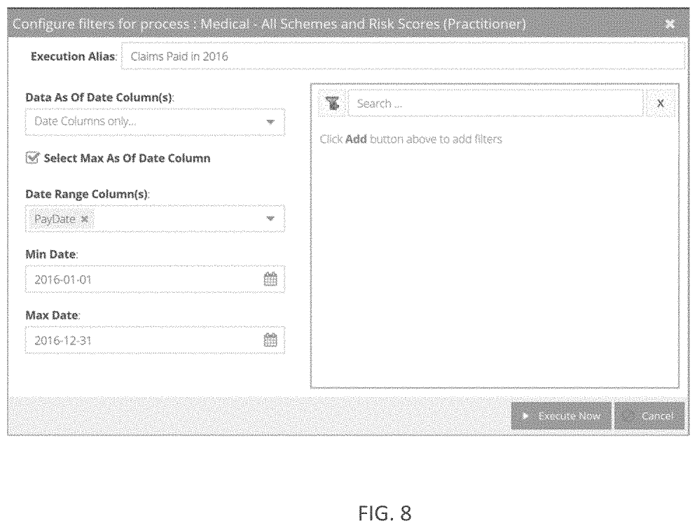

Clicking the play button brings up a menu where the user can name the execution, configure a date window (based on any available date column), and/or configure a filter, for example, as shown in FIG. 8.

These filters filter all available data sources that contain the chosen column for the value(s) selected before the given step in the process is executed. In this example, execution is filtering the input data sources that contain the column “Pay Date” for any value between Jan. 1, 2016 and Dec. 31, 2016.

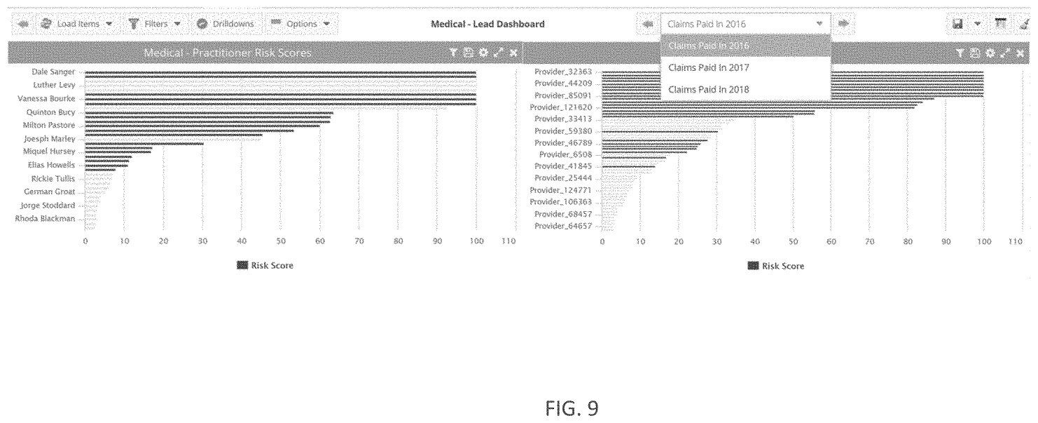

After the process finishes, all dashboards that contain data sources that contain the results of this execution will have the execution's name selectable in the drop-down menu, for example, as shown in FIG. 9 below.

This filter is referred to herein as the Galactic Filter, as the filtration carries over for the Dashboard to Dashboard Drill Down as described above, which effectively allows for versioning between the network of dashboards described in the Dashboard to Dashboard Drill Down section. This is different than the Global Filter and Local Filter, which filter a single dashboard and a single chart respectively.

### Taskboard Down

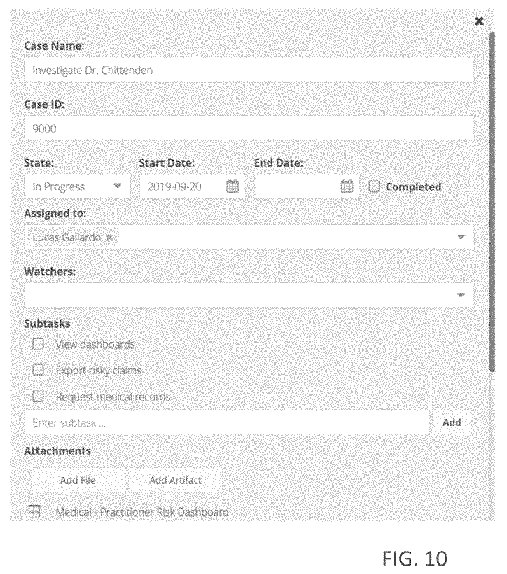

In exemplary embodiments, the Workflow tool allows for the attachment of any Absolute Insight object. FIG. 10 shows an example of the attachment of the Dashboard Medical—Practitioner Risk Dashboard to a task.

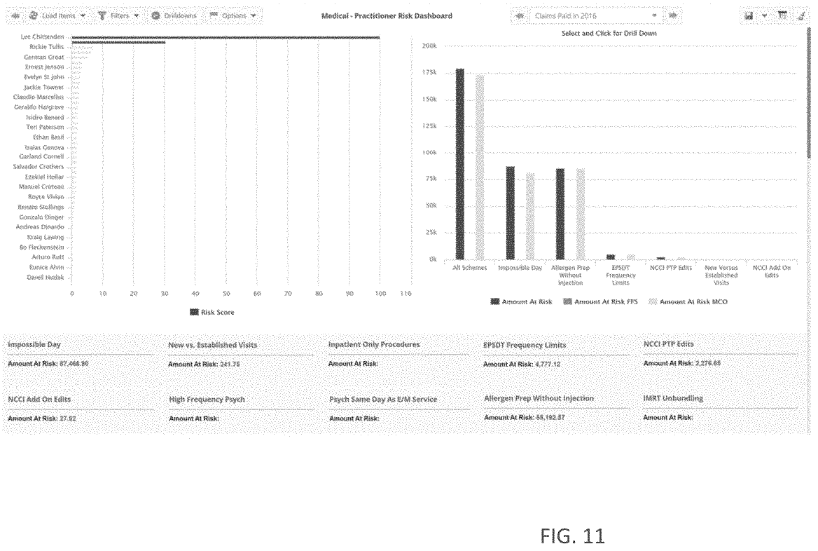

When the dashboard is attached, the application checks to see if the same dashboard is open in the Dashboard tab and, if so, saves a copy of the opened version to the Task, for example, as shown in FIG. 11.

The user can double-click on the dashboard associated with the task and immediately open up the same view of the dashboard the user saw when they attached it to the task.

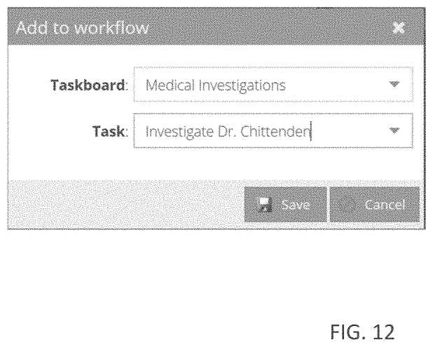

The same can be accomplished by clicking the top-right panel icon. This allows the user to choose an associated task and save the version of the dashboard opened to the associated task, for example, as shown in FIG. 12.

### Micro Services

Micro Services are self-contained packages that are language-independent. In certain exemplary embodiments, the system puts a container (i.e., Application Program Interface or API) around the service so that the system can run it. In this way, it doesn't matter what language is used to code the models. This allows us to be able to pass things around from place to place, i.e., by standardizing the interface.

### Miscellaneous

It should be noted that headings are used above for convenience and are not to be construed as limiting the present invention in any way.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads.

Importantly, it should be noted that embodiments of the present invention may employ conventional components such as conventional computers (e.g., off-the-shelf PCs, mainframes, microprocessors), conventional programmable logic devices (e.g., off-the shelf FPGAs or PLDs), or conventional hardware components (e.g., off-the-shelf ASICs or discrete hardware components) which, when programmed or configured to perform the non-conventional methods described herein, produce non-conventional devices or systems. Thus, there is nothing conventional about the inventions described herein because even when embodiments are implemented using conventional components, the resulting devices and systems (e.g., the FWA analytics system) are necessarily non-conventional because, absent special programming or configuration, the conventional components do not inherently perform the described non-conventional functions.

The activities described and claimed herein provide technological solutions to problems that arise squarely in the realm of technology. These solutions as a whole are not well-understood, routine, or conventional and in any case provide practical applications that transform and improve computers and computer routing systems.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:

P1. A method of processing a claim for a medical service associated with a patient, the method comprising: obtaining at least one medical record associated with the claim; validating, using the electronic medical record, that the service was performed, medically indicated, and consistent with medical norms; and outputting an indication of potential claim fraud if the service was not performed, was not medically indicated, or was not consistent with medical norms.

P2. A method according to claim P1, wherein validating comprises: creating a mathematical model of the patient; and analyzing whether the medical service comports with the mathematical model.

P3. A method according to claim P2, wherein analyzing comprises: determining a degree to which the medical service comports with the mathematical model; and outputting a score indicating the degree.

P4. A method of processing medical claim data, the method comprising: using a FOREL algorithm to categorize the data into a plurality of clusters; and processing the data based on the clusters.

P5. A method according to claim P4, wherein the FOREL algorithm produces a first number of clusters, and wherein processing the data based on the clusters comprises performing an iterative process to dimensionally reduce the number of clusters to be less than the first number of clusters.

P6. A method of processing and visualizing medical claim data, the method comprising: categorizing the data into a plurality of clusters having a first number of clusters; performing an iterative process to dimensionally reduce the number of clusters to a second number of clusters less than the first number of clusters; and producing a visualization based on the second number of clusters.

P7. A method according to claim P6, wherein categorizing the data in a plurality of clusters comprises using a FOREL algorithm to categorize the data into the plurality of clusters.

P8. A method of managing dashboards in a medical claim processing system, the method comprising providing access to a plurality of related dashboards, wherein, from each dashboard, a user can drill down to a lower-level dashboard so as to produce a hierarchy of dashboards.

P9. A method according to claim P8, wherein the user can select an entity on a first dashboard to drill down to a lower-level dashboard, and wherein the lower-level dashboard will be filtered based at least in part on the selected entity.

P10. A method according to claim P8, further comprising: associating the hierarchy of dashboards with a task; and allowing the task to be re-run based on a different set of parameters, wherein all of the dashboards in the hierarchy will be re-run based on the different set of parameters.

P11. A method according to claim P8, further comprising storing the hierarchy of dashboards along with associated data to allow for recall and replay of the dashboards.

P12. A method of processing medical claim data, the method comprising enriching the medical claim data by creating new data points and categories that can be used in the analytics. to P13. A method according to claim P12, wherein enriching comprises: computing and storing distance information, e.g., distance of a patient to a doctor or pharmacy based on latitude/longitude of addresses; and using such distance information in analytics, e.g., the system might flag as suspicious a patient traveling a large distance to a particular doctor or pharmacy, particularly when certain types of activities are involved, e.g., opioid prescriptions.

P14. A method according to claim P12, wherein enriching comprises: creating summary categories, e.g., does a claim involve an opioid (e.g., opioid yes or no), does a claim involve an ADHD medication (e.g., ADHD yes or no), does a claim involve a brand vs. generic medication, etc.; and using such summary categories in analytics, e.g., to simplify certain analyses, e.g., multiple claims involving opioids can be viewed as being similar even if they involve different opioids in different doses of both generics and name-brands where otherwise the claims might appear to be dissimilar.

P15. A medical fraud, waste, and abuse analytics system comprising a processor programmed, via a computer program stored in a tangible, non-transitory computer-readable medium, to perform any one or more of the methods of claims P1-P14.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

What is claimed is:

1. A method of processing a medical claim, the method comprising:
receiving a medical claim associated with a patient;
creating a digital twin of the patient;
mathematically analyzing whether the medical claim comports with the digital twin; and
outputting an indication of potential claim fraud if the medical claim does not comport with the digital twin.

2. A method according to claim 1, wherein mathematically analyzing whether the medical claim comports with the digital twin comprises:
determining a degree to which the medical service comports with the mathematical model; and
outputting a score indicating the degree.

3. A method according to claim 1, wherein the digital twin of the patient is created at least in part using a baseline representation of medical codes to mirror a health state of the patient.

4. A method according to claim 1, wherein the medical claim includes a Resource Utilization Group (RUG) categorization for the patient, and wherein mathematically analyzing whether the medical claim comports with the digital twin comprises:
mathematically analyzing whether the Resource Utilization Group (RUG) categorization comports with the mathematical model.

5. A method according to claim 4, wherein mathematically analyzing whether the Resource Utilization Group (RUG) categorization comports with the digital twin comprises:
determining a projected RUG categorization for the patient based on the digital twin; and
determining whether the projected RUG comports with the RUG in the medical claim.

6. A method according to claim 1, wherein mathematically analyzing whether the medical claim comports with the digital twin comprises:
determining whether a medical service associated with the medical claim was medically indicated based on the digital twin.

7. A method according to claim 1, wherein mathematically analyzing whether the medical claim comports with the digital twin comprises:
determining whether a medical service associated with the medical claim is consistent with medical norms with respect to a health state of the patient.

8. A system for processing a medical claim, the system comprising:
at least one processor coupled to at least one memory storing computer program instructions which, when executed by the at least one processor, cause the system to perform computer processes comprising:
receiving a medical claim associated with a patient;
creating a digital twin of the patient;
mathematically analyzing whether the medical claim comports with the digital twin; and
outputting an indication of potential claim fraud if the medical claim does not comport with the digital twin.

9. A system according to claim 8, wherein mathematically analyzing whether the medical claim comports with the digital twin comprises:
determining a degree to which the medical service comports with the mathematical model; and
outputting a score indicating the degree.

10. A system according to claim 8, wherein the digital twin of the patient is created at least in part using a baseline representation of medical codes to mirror a health state of the patient.

11. A system according to claim 8, wherein the medical claim includes a Resource Utilization Group (RUG) categorization for the patient, and wherein mathematically analyzing whether the medical claim comports with the digital twin comprises:
mathematically analyzing whether the Resource Utilization Group (RUG) categorization comports with the mathematical model.

12. A system according to claim 11, wherein mathematically analyzing whether the Resource Utilization Group (RUG) categorization comports with the digital twin comprises:
determining a projected RUG categorization for the patient based on the digital twin; and
determining whether the projected RUG comports with the RUG in the medical claim.

13. A system according to claim 8, wherein mathematically analyzing whether the medical claim comports with the digital twin comprises:
determining whether a medical service associated with the medical claim was medically indicated based on the digital twin.

14. A system according to claim 8, wherein mathematically analyzing whether the medical claim comports with the digital twin comprises:
determining whether a medical service associated with the medical claim is consistent with medical norms with respect to a health state of the patient.

15. A computer program product comprising a tangible, non-transitory computer readable medium having embodied therein computer program instructions for processing a medical claim which, when executed by at least one computer processor, cause the computer processor to perform computer processes comprising:
receiving a medical claim associated with a patient;
creating a digital twin of the patient;
mathematically analyzing whether the medical claim comports with the digital twin; and
outputting an indication of potential claim fraud if the medical claim does not comport with the digital twin.

16. A system according to claim 15, wherein mathematically analyzing whether the medical claim comports with the digital twin comprises:
determining a degree to which the medical service comports with the mathematical model; and
outputting a score indicating the degree.

17. A system according to claim 15, wherein the digital twin of the patient is created at least in part using a baseline representation of medical codes to mirror a health state of the patient.

18. A system according to claim 15, wherein the medical claim includes a Resource Utilization Group (RUG) categorization for the patient, and wherein mathematically analyzing whether the medical claim comports with the digital twin comprises:
mathematically analyzing whether the Resource Utilization Group (RUG) categorization comports with the mathematical model.

19. A system according to claim 18, wherein mathematically analyzing whether the Resource Utilization Group (RUG) categorization comports with the digital twin comprises:
determining a projected RUG categorization for the patient based on the digital twin; and
determining whether the projected RUG comports with the RUG in the medical claim.

20. A system according to claim 15, wherein mathematically analyzing whether the medical claim comports with the digital twin comprises:
determining whether a medical service associated with the medical claim was medically indicated based on the digital twin.

21. A system according to claim 15, wherein mathematically analyzing whether the medical claim comports with the digital twin comprises:
determining whether a medical service associated with the medical claim is consistent with medical norms with respect to a health state of the patient.
